SEQUENCE LISTING (19 sequences)

SEQ ID NO: 1 - protein, 281 aa, Homo sapiens [sequence omitted]

SEQ ID NO: 2 - protein, 333 aa, Artificial Sequence
  super-TRAIL AX-1611 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 3 - protein, 334 aa, Artificial Sequence
  super-TRAIL AX-1621 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 4 - protein, 334 aa, Artificial Sequence
  super-TRAIL AX-1631 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 5 - protein, 335 aa, Artificial Sequence
  super-TRAIL AX-1622 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 6 - protein, 336 aa, Artificial Sequence
  super-TRAIL AX-1623 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 7 - protein, 336 aa, Artificial Sequence
  super-TRAIL AX-1630 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGGGGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 8 - protein, 337 aa, Artificial Sequence
  super-TRAIL AX-1632 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGGGGGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 9 - protein, 339 aa, Artificial Sequence
  super-TRAIL AX-1618 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGSGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 10 - protein, 343 aa, Artificial Sequence
  super-TRAIL AX-1620 protein sequence
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGSGGGSGGVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 11 - protein, 335 aa, Artificial Sequence
  super-TRAIL AX-1606 protein sequence
QRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGSGGGGSGGQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 12 - protein, 6 aa, Artificial Sequence
  Synthetic Construct
  VARIANT 1..6: Can be present in repeats of any integer
GGGGGS

SEQ ID NO: 13 - protein, 5 aa, Artificial Sequence
  Synthetic Construct
  VARIANT 1..5: Can be present in repeats of any integer
GGGGS

SEQ ID NO: 14 - protein, 4 aa, Artificial Sequence
  Synthetic Construct
  VARIANT 1..4: Can be present in repeats of any integer
GGGS

SEQ ID NO: 15 - protein, 3 aa, Artificial Sequence
  Synthetic Construct
  VARIANT 1..3: Can be present in repeats of any integer
GGS

SEQ ID NO: 16 - protein, 2 aa, Artificial Sequence
  Synthetic Construct
  VARIANT 1..2: Can be present in repeats of any integer
GS

SEQ ID NO: 17 - protein, 4 aa, Artificial Sequence
  Synthetic Construct
  VARIANT 1..4: Can be present in repeats of any integer
GGSS

SEQ ID NO: 18 - protein, 6 aa, Artificial Sequence
  Synthetic Construct
  VARIANT 1..6: Can be present in repeats of any integer
GGGSSS

SEQ ID NO: 19 - protein, 4 aa, Artificial Sequence
  Synthetic Construct
  VARIANT 1..4: Can be present in repeats of any integer
GSSS

(19) United States
(12) Patent Application Publication
(10) Pub. No.: US 2024/0254193 A1
(43) Pub. Date: Aug. 1, 2024

(54) A SUPER-TRAIL MOLECULE COMPRISING TWO TRAIL TRIMERS

(71) Applicant: Bejing Anxinhuaide Biotech, Co., Ltd, Beijing (CN)

(72) Inventors: Xinguo QIAN, Beijing (CN); Wei HONG, Beijing (CN)

(73) Assignee: Bejing Anxinhuaide Biotech, Co., Ltd, Beijing (CN)

(21) Appl. No.: 18/564,554

(22) PCT Filed: May 27, 2022
(86) PCT No.: PCT/CN2022/095472

§ 371 Date: Nov. 27, 2023

(30) Foreign Application Priority Data
May 27, 2021 (WO) PCT/CN2021/096498

(51) Int. Cl. C07K 14/705; A61K 38/00; A61P 35/00; C07K 14/525

(52) U.S. Cl. CPC C07K 14/70575; A61P 35/00; C07K 14/525; A61K 38/00; C07K 2319/30; C07K 2319/31

(57) ABSTRACT

The present invention demonstrates a fusion protein comprising the human TRAIL extracellular domain, a flexible linker, the second human TRAIL extracellular domain from N-terminus to C-terminus. The fusion protein, termed as “super-TRAIL”, forms a hexamer that contains two TRAIL trimers in solution; the fusion protein exhibits greatly improved biological activity to induce apoptosis in cancer cells compared with the wild type human TRAIL.

### CROSS REFERENCE TO RELATED APPLICATIONS

This application claims priority from PCT international application PCT/CN2021/096498 filed on May 27, 2021, which is incorporated herein by reference in its entirety.

### FIELD OF THE INVENTION

The present invention discloses a recombinant fusion protein which exhibits the potent biological activity to induce apoptosis in cancerous cells. The fusion protein may be utilized as a biological drug to treat multiple cancers.

### BACKGROUND

TNF-related apoptosis inducing ligand (TRAIL) gene was first cloned and named by Wiley, et al. in 1995[1]. In 1996, the same gene was cloned and named as Apo2L [2]. The human TRAIL gene encodes a protein containing a cytoplasmic tail, a transmembrane region and the extracellular domain from N-terminus to C-terminus. The TRAIL extracellular domain can also be released from cell membrane by proteolytic cleavage. Both the full-length membrane-bound TRAIL and the soluble TRAIL extracellular domain form stable homotrimers and bind their receptors to perform biological effects. A large number of in vivo and in vitro experiments show that TRAIL can selectively induce apoptosis of many tumor cells and transformed cells. Application of recombinant TRAIL protein in tumor-bearing animals can significantly inhibit tumor cell growth and even result in tumor regression without obvious damage to the host.

TRAIL belongs to the tumor necrosis factor (TNF) superfamily which is a type II membrane protein containing a TNF homology extracellular domain and forms stable homo-trimer in solution. The TNF superfamily members regulate a wide range of cell functions including immune response and inflammation, but also proliferation, differentiation, apoptosis and embryogenesis. The TNF superfamily contains 19 members and they function by binding to the TNF receptor superfamily members.

TRAIL can bind its receptors TRAIL-R1(DR4) and TRAIL-R2(DR5) to cluster three receptors around the TRAIL homo-trimer to initiate the down stream signaling. It has been reported that co-administration of antibody against DR5 with TRAIL can significantly increase the activity of TRAIL, possibly by further crosslinking the TRAIL receptor clusters [3]. Moreover, the hyper-oligomerized TRAIL by addition of DTT showed much enhanced activity to kill cancer cells in vitro [4]. In this invention, we disclose a fusion protein that contains the human TRAIL extracellular domain, a flexible linker, the second human TRAIL extracellular domain from N-terminus to C-terminus. We termed this fusion protein as “super-TRAIL” because this fusion protein exhibits greatly improved biological activity to induce apoptosis in cancer cells compared with the wild type human TRAIL

### SUMMARY

In one aspect, the present disclosure provides a fusion polypeptide comprising the first human TRAIL extracellular domain, a flexible linker, the second human TRAIL extracellular domain from N-terminus to C-terminus; the fusion polypeptide forms a hexamer that contains two TRAIL trimers in solution; the fusion polypeptide exhibits greatly improved biological activity to induce apoptosis in cancer cells compared with the wild type human TRAIL.

In some embodiments, the fusion polypeptide demonstrates an improved in vivo plasma half-life compared with the wild type human TRAIL.

In some embodiments, the human TRAIL extracellular domains may be selected but not limited from TRAIL residues 114-281, TRAIL residues 118-281, TRAIL residues 119-281, TRAIL residues 120-281, or TRAIL residues 122-281; and the first TRAIL extracellular domain and the second TRAIL extracellular domain may be the same or different.

In some embodiments, the fusion polypeptide has the protein sequence selected from SEQ ID NOs: 2 to 11.

In another aspect, the present disclosure provides a fusion protein comprising the above fusion polypeptide. In some embodiments, the fusion protein includes but not limited to IgG Fc fusion proteins containing the fusion polypeptide and HAS (human serum albumin) fusion proteins containing the fusion polypeptide.

In some embodiments, the fusion polypeptide may be any modified polypeptides, wherein the modification includes but not limited to PEGylation, lipidation and glycosylation; the modified fusion polypeptide may exhibit extended in vivo plasma half life or reduced immunogenicity.

In another aspect, the present disclosure provides a polypeptide with an amino sequence being at least 90% sequence homology to the fusion polypeptide.

In another aspect, the present disclosure provides a polynucleotide sequence encoding the fusion polypeptide or the fusion protein.

In another aspect, the present disclosure provides a pharmaceutical composition comprising the fusion polypeptide or the fusion protein and a physiologically acceptable excipient.

In another aspect, the present disclosure provides a method for generating a more biologically potent TNF (tumor necrosis factor) superfamily member comprising connecting two TNF superfamily member molecules via a flexible linker to constitute a hexamer of two trimers of the TNF family member; and the TNF family members include but not limited to TNF, 4-1BBL, Fas ligand, OX40L, CD40L, CD256, CD257, CD258 and GITRL.

In another aspect, the present disclosure provides use of the fusion polypeptide or the fusion protein in the manufacture of a medicament for the treatment of a cancer.

In some embodiments, the cancer is a multiple myeloma or a lung cancer.

In another aspect, the present disclosure provides a method of treating a cancer comprising administrating to a subject in need thereof a therapeutically effective amount of the fusion polypeptide, the fusion protein, or the pharmaceutical composition.

In some embodiments, the cancer is a multiple myeloma or a lung cancer. The present invention demonstrates a “super-TRAIL” molecule comprising two human TRAIL extracellular domains connected by a flexible linker. The recombinant super-TRAIL protein shows greatly enhanced activity to induce apoptosis in cancer cells compared with the wild type TRAIL. Because of the increased molecular weight, the super-TRAIL protein exhibits the improved pharmacokinetic profile when administered into a subject compared to the wild type TRAIL.

### BRIEF DESCRIPTION OF THE DRAWINGS

FIG. 1. The schematic drawings of the super-TRAIL molecule. A) the wild type TRAIL homo-trimer. The TRAIL monomers are shown by ovals in red, green and blue. The N-terminus and C-terminus of one monomer is labeled. B) The super-TRAIL molecule containing a TRAIL hexamer of two trimers formed by use of the stacking model. The super-TRAIL monomers comprise two TRAIL extracellular domains and are shown in red, green and blue. In this model, the two TRAIL trimers are connected via three flexible linkers and two trimers are positioned stacking on each other. Therefore the two trimers are positioned in anti-parallel fashion. C) The super-TRAIL molecule containing a TRAIL hexamer of two trimers folded by the side-by-side model. In this model, the two TRAIL trimers are connected via one flexible linker and the two trimers are placed side by side. The two trimers are placed in parallel fashion.

FIG. 2. Super-TRAIL molecule forms a hexamer of two TRAIL trimers in solution. A) the gel filtration profiles of superTRAIL AX-1611 and wild type TRAIL. Both of the purified proteins were loaded on gel filtration column Superdex200. The molecular weights of the protein standards are labeled by arrows. The apparent molecular weight of AX-1611 estimated from the profile was ˜96 kD. The gel filtration chromatography indicated super-TRAIL AX-1611 contains three monomers that comprise six TRAIL extracellular domains. The vertical axis indicates the OD280 readings and the horizontal axis shows the elution volume (ml). B) the negative stain electron microscope studies of super-TRAIL AX-1611. The top panel shows one of the raw images from the negative stain electron microscopy. Some of the peanut-like particles are indicated by arrows. The lower panel shows the top picks from the 2D classification of the negative stain images by use of the software Relion. C) The 3D reconstruction of the super-TRAIL AX-1611. Two of the human TRAIL homo-trimer structures can be fitted into the AX-1611 molecule generated by Relion. In the structure, the two TRAIL trimers may be associated together via side-by-side model. The two TRAIL trimers are connected by a single flexible linker that is clearly shown in the structure. The two trimers are placed in parallel fashion in the structure. AX-1611 molecule contains three AX-1611 monomers that are shown in red, green and blue (similar as that in FIG. 1C).

FIG. 3. The in vitro biological activities of AX-1611. The horizontal axis indicates the protein concentrations and the vertical axis shows the OD490 from MTS assay. A) The biological activities of AX-1611 and wild type TRAIL (wtTRAIL) to induce apoptosis in mouse L929 cells. The error bars indicate the standard derivations of three independent experiments. B) The biological activities of AX-1611 and wild type TRAIL to induce apoptosis in human H460 cells. C) The biological activities of AX-1611 and wild type TRAIL to induce apoptosis in human RPMI-8226 cells.

FIG. 4: The in vivo anti-tumor activity of the super-TRAIL AX-1611 and wildtype Trail. Nude mice with established RPMI-8226 xenografts were given AX-1611 (2, 10 mg/kg/day i.p.) or wildtype Trail (10 mg/kg/day, i.p.) for consecutive ten days (n=5/group). Results shown are group mean (±S.D). The horizontal axis indicates days after the first treatment.

FIG. 5: The in vivo anti-tumor activity of the super-TRAIL AX-1611 and wildtype Trail. Nude mice with established NCI-H460 xenografts were given AX-1611 (10 mg/kg/day, i.p.) or wildtype Trail (10 mg/kg/day i.p.) or PBS for consecutive ten days (n=5/group). Results shown are group mean (±S.D). The horizontal axis indicates days after the first treatment.

### DETAILED DESCRIPTION

### Terms Used in this Invention

The articles “a”, “an” and “the” are used herein to refer to one or to more than one (i.e., to at least one) of the grammatical object of the article. By way of example, “an element” means one element or more than one element.

The term “TRAIL”, “native TRAIL”, “wild-type TRAIL” or “wtTRAIL” indicates TNF-related apoptosis inducing ligand. TRAIL has alternative names such as Apo2L, CD253 or TNFSF10. The native TRAIL forms a homo-trimer in solution. The sequence of the human TRAIL is shown in SEQ ID NO: 1.

The term “flexible polypeptide linker” refers to an amino acid sequence which is flexible in movement and which does not form any regular stable secondary and tertiary protein structures. The terms “flexible polypeptide linker”, “flexible linker”, “flexible unstructured polypeptide”, “flexible unstructured polypeptide sequence”, and “flexible unstructured linker” “flexible unstructured polypeptide linker” are used interchangeably in this invention.

The term “IgG” herein indicates the antibody Immunoglobin G. The IgG proteins contain two identical heavy chains and two identical side chains.

The term “Fc moiety”, “Fc domain” or “Fc region” herein is used to define a C-terminal region of an antibody IgG heavy chain. The term includes native sequence Fc regions and variant Fc regions. An IgG Fc region comprises the hinge region, an IgG CH2 and an IgG CH3 domain.

The term “EC₅₀”, also known as half maximal effective concentration, refers to the concentration of a protein or a drug that gives half-maximal response.

The phrase “pharmaceutically acceptable excipient” as used herein refers to a pharmaceutically acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting a therapeutic compound for administration to a subject. Each excipient should be “acceptable” in the sense of being compatible with the other ingredients of the formulation and not injurious to the subject.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the dosing regimen to be followed, whether it is administered in combination with other therapeutics, timing of administration, the tissue to be imaged, and the physical delivery system in which it is carried.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The terms “cancer” as used herein refers to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, lung adenocarcinoma, head/neck squamous cell cancer, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, rectal cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, bone cancer, Ewing's sarcoma, cervical cancer, brain cancer, stomach cancer, bladder cancer, hepatoma, breast cancer, colon carcinoma, uterine cancer, ovarian cancer, and head and neck cancer.

The term “sequence identity” in the context of two or more peptides, is defined as the percentage of amino acid residues in a candidate sequence that are identical with the amino acid residues in the reference peptide or antibody sequence, after aligning the sequences and introducing gaps, if necessary, to achieve the maximum correspondence over a comparison window or designated region. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. In a specific embodiment, the sequence identity is acquired through BLAST software that is publicly available on the worldwide web at ncbi.nlm.nih.gov, with default parameters.

### Design of the Biologically Potent Super-TRAIL Molecule

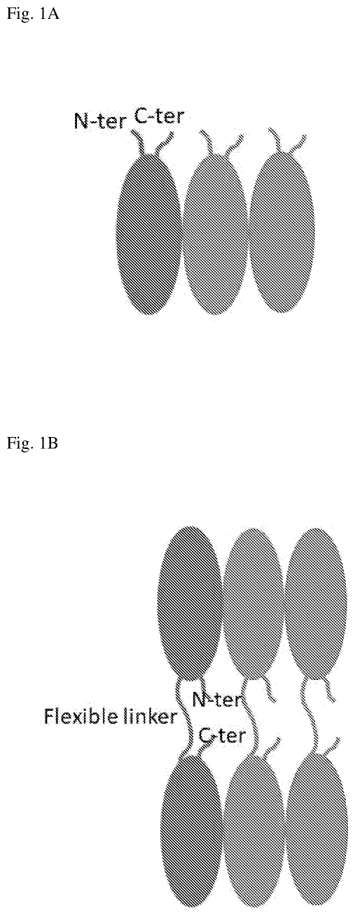

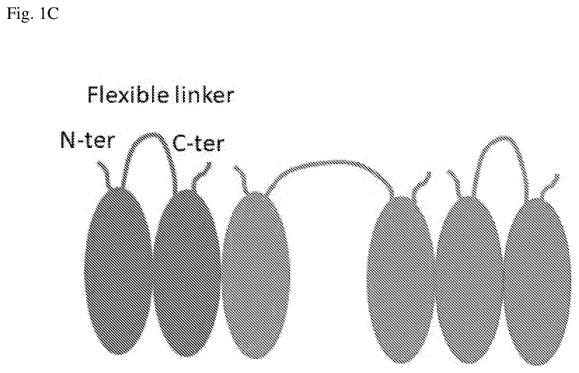

The TRAIL homo-trimer initiates the apoptosis signaling pathway by associating three TRAIL receptors together. Convincing data have demonstrated that higher order oligomerization of the TRAIL receptor DR4 or DR5 may generate stronger signals for apoptosis. In this invention, we designed a super-TRAIL protein comprising the human TRAIL extracellular domain, a flexible linker, the second human TRAIL extracellular domain from N-terminus to C-terminus. Because each TRAIL monomer forms stable trimer, three super-TRAIL polypeptide chains will simultaneously fold into two connected TRAIL trimers. The super-TRAIL molecule may contain a TRAIL hexamer of two trimers formed by use of either the stacking model or the side-by-side model (FIG. 1). Within the super-TRAIL protein, the flexible linker between the two TRAIL extracellular domains may provide ample flexibility for the correct protein folding. We reasoned that the super-TRAIL may exhibit potent biological activity to induce apoptosis by interacting with six TRAIL receptors simultaneously.

### Super-TRAIL Molecule Forms a Hexamer of Two TRAIL Trimers in Solution

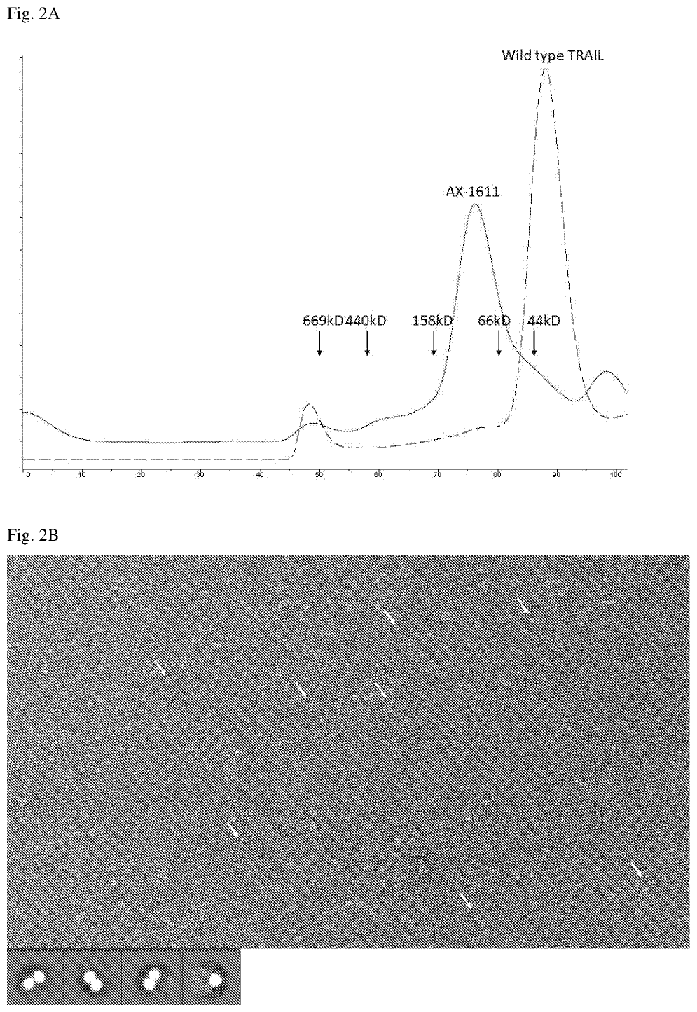

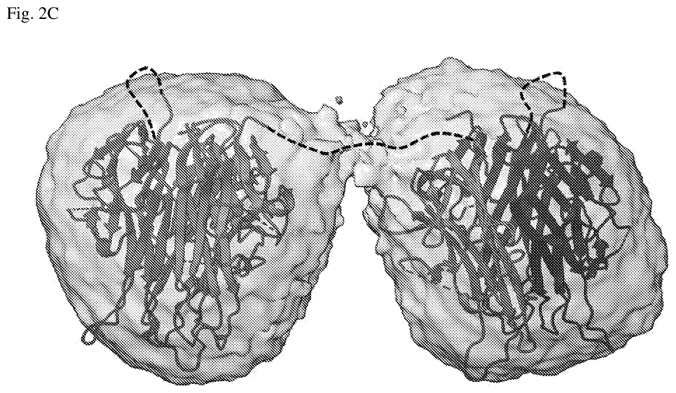

In some embodiments of the invention, we generated a super-TRAIL molecule AX-1611 containing human TRAIL residues 114-281, a linker of five glycine residues and human TRAIL residues 122-281. The sequence of the super-TRAIL AX-1611 is shown in SEQ ID NO: 2. The recombinant super-TRAIL AX-1611 was expressed and purified to homogeneity. The purified AX-1611 was loaded on the gel filtration column superdex200, the apparent molecular weight of AX-1611 estimated from the elution profile was ˜96 kD (FIG. 2). The calculated molecular weight for each AX-1611 monomer is 38 kD, therefore the AX-1611 molecule may contain three monomers. Because each AX-1611 monomer is composed of two TRAIL extracellular domains, the AX-1611 molecule may comprise six TRAIL extracellular domains.

The purified AX-1611 was further examined by negative stain electron microscopy. The negative stain images indicated that many AX-1611 molecules appeared as peanut-like particles (FIG. 2). The 3D reconstruction of the negative stain images by use of the software Relion provided the molecular shape of AX-1611 at the resolution of ˜25 Å. Two of the human TRAIL homo-trimer structures can be nicely fitted into the AX-1611 molecule by Relion. The structure clearly indicates that the two TRAIL trimers within AX-1611 are connected by a single flexible linker (FIG. 2). In the structure, three AX-1611 monomers can be folded into two TRAIL trimers and the two TRAIL trimers are associated together via side-by-side model. The two TRAIL trimers within AX-1611 are aligned in the parallel fashion (not anti-parallel fashion) which allows the super-TRAIL to conveniently interact with six TRAIL receptors. Biochemical and biophysical data demonstrate that the super-TRAIL AX-1611 contains a hexamer of the TRAIL extracellular domains that are arranged into two trimers by use of the side-by-side model.

Super-TRAIL Exhibits Much Enhanced Activity to Induce Apoptosis in Cancer Cells Compared with Wild Type TRAIL

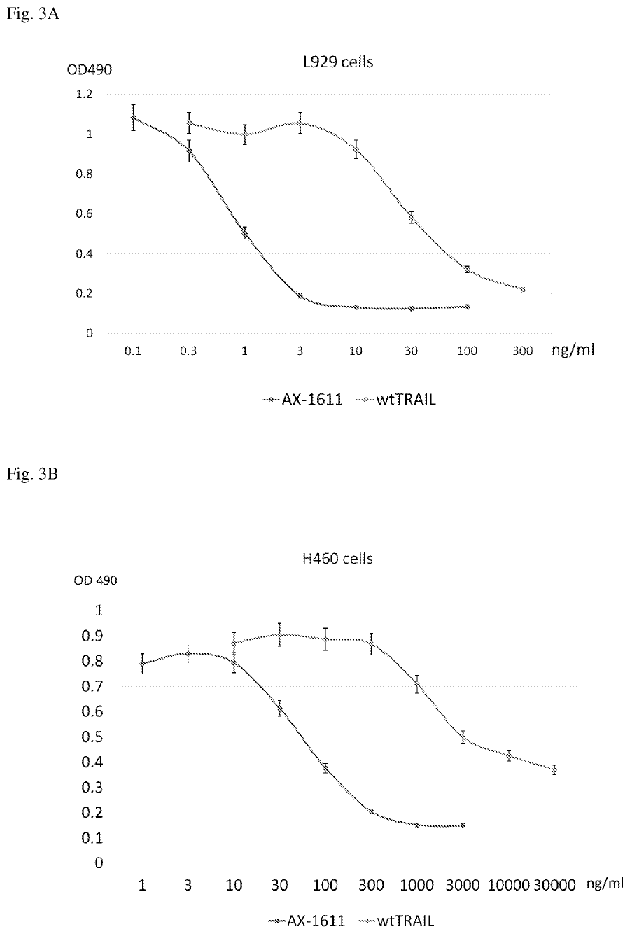

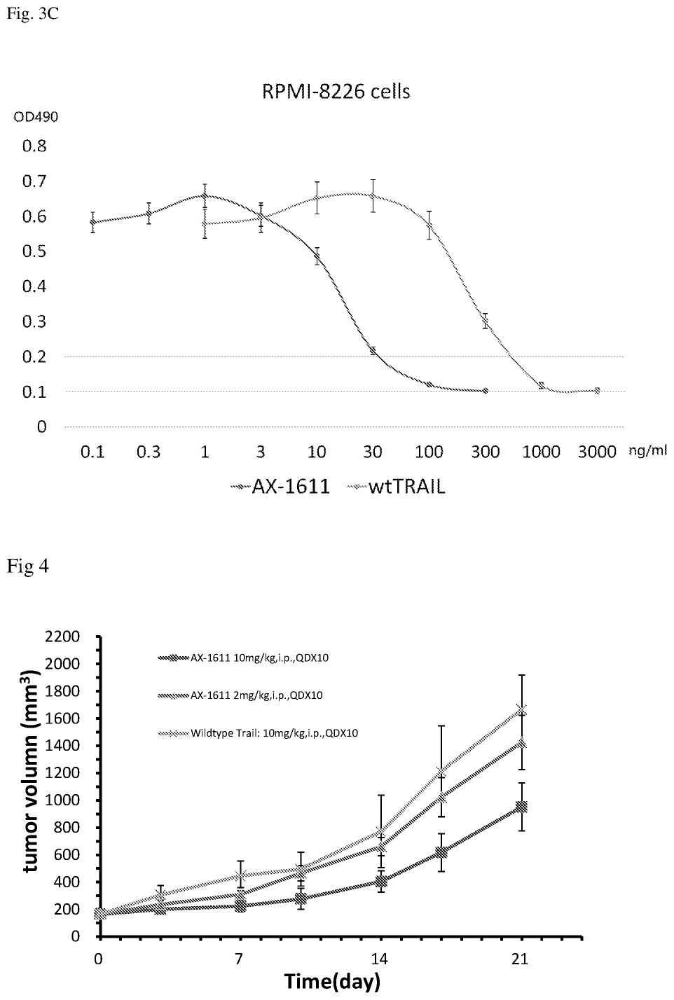

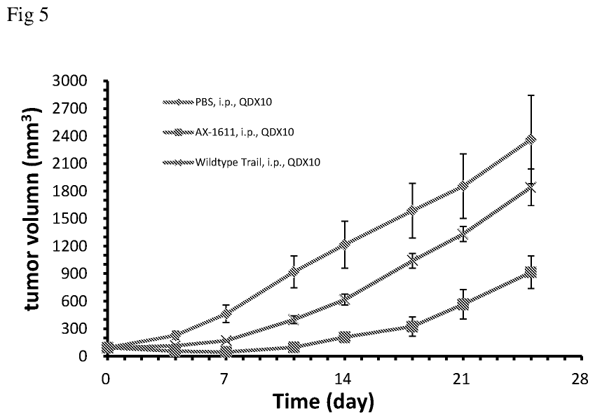

We examined the in vitro biological activity of the super-TRAIL AX-1611 to induce apoptosis in multiple cell lines. The mouse L929 cell line has been utilized as the standard cells to test the TRAIL activity. The super-TRAIL AX-1611 exhibited ˜30 fold enhanced activity to induce apoptosis in L929 cells compared with wild type TRAIL (FIG. 3). In human lung cancer cell line H460 and human multiple myeloma cell line RPMI-8226, super-TRAIL AX-1611 also demonstrated much stronger activity compared with wild type TRAIL (FIG. 3). Also we examined the in vivo anti-tumor activity of the super-TRAIL AX-1611 by using of tumor cell xenograft models. In RPMI-8226 xenograft models, treatment with AX-1611 can lead to significant tumor regression. The data clearly indicated that AX-1611 even at the dosage of 2 mg/kg was more potent in tumor suppression than the wild type TRAIL at the dosage of 10 mg/kg (FIG. 4). The data of NCI-H460 xenograft models showed that AX-1611 exhibited much more potent anti-tumor activity than wild type TRAIL (FIG. 5). We reason that super-TRAIL contains a hexamer of two TRAIL trimers and the super-TRAIL molecule can bind six TRAIL receptors simultaneously. Thus, the multi-valency of super-TRAIL renders the superior biological activity over the wild type TRAIL homo-trimer.

In some embodiments of the invention, we generated a super-TRAIL molecule which shows greatly enhanced activity compared with the wild type TRAIL to induce apoptosis in cancerous cells. The super-TRAIL exhibits the potent activity by itself without additions of other crosslinking reagents such as antibodies or DTT. Moreover, the super-TRAIL molecule contains only the wild type TRAIL sequence and no other foreign sequences. This may provide super-TRAIL low immunogenicity when it is administrated into human. IgG Fc and single-chain TRAIL fusion protein has been generated and it may have better activity than the wild type TRAIL [5]. Our data indicated that the super-TRAIL AX-1611 exhibited ˜5-10 fold more potent activity to induce apoptosis in L929 cells compared with the Fc-single chain TRAIL fusion protein. We reason that the super-TRAIL molecule contains two TRAIL trimers that are well positioned to interact with the TRAIL receptors to induce apoptosis. This may provide super-TRAIL advantages over other fusion proteins.

### The Flexible Linker in the Super-TRAIL Sequence

In the super-TRAIL protein sequence, a flexible polypeptide linker is utilized to connect two TRAIL extracellular domain. In some embodiments of the invention, the flexible polypeptide linkers in the fusion protein are rich in Glycine and Serine residues. In the flexible polypeptide linker sequence, the sum of amino acid residues of G, S, E, A, P and T may constitute more than 90% of the primary sequence; and the flexible polypeptide sequence has greater than 90% unstructured random coil formation as determined by GOR algorithm.

In some embodiments of this invention, the flexible linker may contain sequences including (G₅S)n, (G₄S)n, (G₃S)n, (G₂S)n, (GS)n, (G₂S₂)n, (G₃S₃)n, (GS₃)n where n is an integer. The flexible linker may contain 0 to 100 amino acid residues.

In some preferred embodiments of this invention, the flexible linker may contain 5 to 20 amino acid residues. We have constructed a number of super-TRAIL molecules with the flexible linker lengths ranging from 5-15 amino acid residues. The sequences of these super-TRAIL molecules are shown in SEQ ID NO: 3-10. These super-TRAIL molecules all showed biological activities to induce apoptosis as measured by use of the L929 cell lines (Table 1).

TABLE 1
The in vitro activities of the super-TRAIL molecules

| | EC50 measured by use of L929 cells (ng/ml) |
|---|---|
| Wild type TRAIL | 32 |
| AX-1611 | 0.85 |
| AX-1621 | 0.95 |
| AX-1631 | 1.2 |
| AX-1622 | 5.3 |
| AX-1623 | 0.88 |
| AX-1630 | 0.78 |
| AX-1632 | 1.10 |
| AX-1618 | 1.5 |
| AX-1620 | 2.1 |
| AX-1606 | 1.05 |

The TRAIL Extracellular Domain within the Super-TRAIL Sequence

The extracellular domain of human TRAIL was estimated to be TRAIL residues 39-281 by Uniprot database. It has been reported that TRAIL (114-281) is efficient to induce apoptosis in cancer cells [3]. Other constructs such as TRAIL (95-281), TRAIL (118-281), TRAIL (119-281) and TRAIL (120-281) are all biologically active to induce apoptosis. In this invention, the TRAIL extracellular domain may be selected from any fragment ranging from human TRAIL residues 39-281 that is biological active to induce apoptosis.

In this invention, we generate a super-TRAIL comprising the human TRAIL extracellular domain, a flexible linker, the second human TRAIL extracellular domain from N-terminus to C-terminus. In some embodiments of this invention, the two TRAIL extracellular domains within the super-TRAIL sequence may be identical (SEQ ID NO: 11). In some embodiments of this invention, the two TRAIL extracellular domains within the super-TRAIL are different (SEQ ID NO: 2-10). A skilled artisan may appreciate that various designs of two human TRAIL extracellular domains linked by a flexible linker fall in the range of this invention.

### The Modification of Super-TRAIL

A number of protein modification methods such as PEGylation, lipidation and glycosylation have been developed to extend the in vivo plasma half life or reduce immunogenicity for a protein of interest. A skilled artisan may understand that any modification of the super-TRAIL molecule, which includes but not limited to PEGylation, lipidation and glycosylation is under the coverage of this invention.

Protein fusion with IgG Fc or HAS (human serum albumin) may significantly increase the in vivo half life for a protein of interest. Any fusion protein includes but not limited to IgG Fc fusion proteins containing the super-TRAIL, HAS (human serum albumin) fusion proteins containing the super-TRAIL is within the disclosed coverage of this invention.

### EXAMPLES

The examples described herein are not intended to represent that the experiments below are all or the only experiments performed. Efforts have been made to ensure accuracy with respect to numbers used (for example, amounts, temperature, etc.), but some experimental errors and deviations should be accounted for.

### Example 1. Construction of the Super-TRAIL AX-1611

In this example, we constructed a super-TRAIL molecule AX-1611 containing human TRAIL residues 114-281, a flexible linker of five glycine residues and human TRAIL residues 122-281. The sequence of the super-TRAIL AX-1611 is shown in SEQ ID NO: 2.

The gene encoding AX-1611 sequence was codon optimized and synthesized. The recombinant AX-1611 can be produced using either bacteria expression system or mammalian expression system. The recombinant AX-1611 was expressed and purified to homogeneity.

To analyze the oligomerization status of AX-1611, the purified AX-1611 was loaded on gel filtration column superdex200 in PBS buffer. The apparent molecular weight of AX-1611 estimated from the elution profile was ˜96 kD (FIG. 2). The calculated molecular weight for each AX-1611 monomer is 38 kD, therefore the AX-1611 molecule may contain three monomers. Because each AX-1611 monomer is composed of two TRAIL extracellular domains, the AX-1611 molecule may comprise six TRAIL extracellular domains.

The human wild type TRAIL residues 114-281 was expressed and purified to serve as a control.

### Example 2. Structural Studies of AX-1611 by Negative Stain Electron Microscopy

The purified AX-1611 was further examined by negative stain electron microscopy. The negative stain images indicated that many of the AX-1611 molecules appeared as peanut-like particles (FIG. 2). 2D classification of the negative stain images by the software Relion provided clear classes with the particles of two dots (FIG. 2). 3D reconstruction by averaging of the selected 1509 particles gave the molecular shape of AX-1611 at the resolution of ˜25 Å. Two human TRAIL homo-trimers can be nicely fitted into the AX-1611 structure by use of Relion. The AX-1611 structure derived from electron microscopy clearly indicated that the two TRAIL trimers are associated by the side-by-side model. The two TRAIL trimers are connected by one single linker as shown by the structure. The two TRAIL trimers are aligned in the parallel fashion.

### Example 3. The In Vitro Activities of Super-TRAIL Molecules

The in vitro biological activity of the super-TRAIL AX-1611 to induce apoptosis was examined by use of mouse L9292 cell line. L929 cells were cultured in DMEM medium supplemented by 10% FCS in 5% CO₂ incubator. 1×10⁴ cells were placed into each well of the 96-well plate. 24 hours later, various concentrations of the AX-1611 were added into each well. 1 μg/ml actinomycin D was also added into each well. Wild type TRAIL was utilized as the control in the experiment. 24 hours later, the viability of the L929 cells may be measured by MTS assay (Promega). By use of the data fitting, the EC50 of AX-1611 was calculated to be 0.85 ng/ml while the EC50 of wild type TRAIL was estimated to be 32 ng/ml (FIG. 3). The EC50 values of other super-TRAIL molecules measured by use of L929 cells were listed in table 1.

The ability of AX-1611 to induce apoptosis in human cancer cells was also tested. The human lung cancer cell line H460 and human multiple myeloma cell line RPMI-8226 were cultured in DMEM medium supplemented by 10% FCS in 5% CO₂ incubator. 5000 cells were placed into each well of the 96-well plate. 24 hours later, various concentrations of the AX-1611 were added into each well. Wild type TRAIL was utilized as the control in the experiment. 24 hours later, the viability of the cells may be measured by use of the MTS assay. The EC50 values of AX-1611 for H460 and RPMI-8226 were measured to be 54.7 and 15.4 ng/ml, respectively. On the other hand, the EC50 values of wild type TRAIL for H460 and RPMI-8226 were estimated to be 1400 and 270 ng/ml, respectively (FIG. 3).

### Example 4: The In Vivo Anti-Tumor Activity of the Super-TRAIL AX-1611

The antitumor activity of super-TRAIL AX-1611 was examined by use of the mice carrying RPMI-8226 xenograft models. A total of 107 RPMI-8226 cells were injected subcutaneously into the right flank of female B-NDG mice (5-8 weeks old, 5 mice per group). Tumor size was monitored by measuring the length (a) and width (b) of the tumor with a caliper, and the tumor volume was calculated by the equation of 0.5×a×b2. Treatments were initiated when tumors reached the volume of approximately 160 mm³. AX-1611 was applied intraperitoneally once per day for 10 days at two different doses (2 mg/kg and 10 mg/kg). Control mice received injections of wild type TRAIL at the dose of 10 mg/kg. Treatment with AX-1611 can lead to significant tumor regression at both dosages. The data clearly indicated that AX-1611 even at the dosage of 2 mg/kg was more potent in tumor suppression than the wild type TRAIL at the dosage of 10 mg/kg (FIG. 4).

The antitumor activity of the super-TRAIL AX-1611 was also analyzed by use of the NCI-H460 xenograft models. A total of 2×10⁶ NCI-H460 cells were injected subcutaneously into the right flank of female B-NDG mice (5-8 weeks old, 5 mice per group). Tumor size was monitored by measuring the length (a) and width (b) of the tumors with a caliper, and the tumor volume was calculated by the equation of 0.5×a×b². Treatments were started when tumors reached the volume of approximately 100 mm³. AX-1611 was applied intraperitoneally once per day for 10 days at dose of 10 mg/kg. Control mice received injections of PBS and wild type TRAIL at dose of 10 mg/kg, respectively. The data showed that AX-1611 exhibited much more potent anti-tumor activity than wild type TRAIL in mice carrying NCI-H460 xenograft models (FIG. 5).

Listed below are some amino acid sequences mentioned herein.

full-length human TRAIL protein sequence.
SEQ ID NO: 1
MAMMEVQGGPSLGQTCVLIVIFTVLLQSLCVAVTYVYFTNELKQM
QDKYSKSGIACFLKEDDSYWDPNDEESMNSPCWQVKWQLRQLVRK
MILRTSEETISTVQEKQQNISPLVRERGPQRVAAHITGTRGRSNT
LSSPNSKNEKALGRKINSWESSRSGHSFLSNLHLRNGELVIHEKG
FYYIYSQTYFRFQEEIKENTKNDKQMVQYIYKYTSYPDPILLMKS
ARNSCWSKDAEYGLYSIYQGGIFELKENDRIFVSVTNEHLIDMDH
EASFFGAFLVG,
super-TRAIL AX-1611 protein sequence.
The linker sequence is underlined.
SEQ ID NO: 2
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGGGVAAHITG
TRGRSNTLSSPNSKNEKALGRKINSWESSRSGHSFLSNLHLRNGE
LVIHEKGFYYIYSQTYFRFQEEIKENTKNDKQMVQYIYKYTSYPD
PILLMKSARNSCWSKDAEYGLYSIYQGGIFELKENDRIFVSVTNE
HLIDMDHEASFFGAFLVG,
super-TRAIL AX-1621 protein sequence.
The linker sequence is underlined.
SEQ ID NO: 3
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGSGGVAAHIT
GTRGRSNTLSSPNSKNEKALGRKINSWESSRSGHSFLSNLHLRNG
ELVIHEKGFYYIYSQTYFRFQEEIKENTKNDKQMVQYIYKYTSYP
DPILLMKSARNSCWSKDAEYGLYSIYQGGIFELKENDRIFVSVTN
EHLIDMDHEASFFGAFLVG,
super-TRAIL AX-1631 protein sequence.
The linker sequence is underlined.
SEQ ID NO: 4
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGGGGVAAHIT
GTRGRSNTLSSPNSKNEKALGRKINSWESSRSGHSFLSNLHLRNG
ELVIHEKGFYYIYSQTYFRFQEEIKENTKNDKQMVQYIYKYTSYP
DPILLMKSARNSCWSKDAEYGLYSIYQGGIFELKENDRIFVSVTN
EHLIDMDHEASFFGAFLVG,
super-TRAIL AX-1622 protein sequence.
The linker sequence is underlined.
SEQ ID NO: 5
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGSGGGVAAHI
TGTRGRSNTLSSPNSKNEKALGRKINSWESSRSGHSFLSNLHLRN
GELVIHEKGFYYIYSQTYFRFQEEIKENTKNDKQMVQYIYKYTSY
PDPILLMKSARNSCWSKDAEYGLYSIYQGGIFELKENDRIFVSVT
NEHLIDMDHEASFFGAFLVG,
super-TRAIL AX-1623 protein sequence.
The linker sequence is underlined.
SEQ ID NO: 6
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGSGGGGVAAH
ITGTRGRSNTLSSPNSKNEKALGRKINSWESSRSGHSFLSNLHLR
NGELVIHEKGFYYIYSQTYFRFQEEIKENTKNDKQMVQYIYKYTS
YPDPILLMKSARNSCWSKDAEYGLYSIYQGGIFELKENDRIFVSV
TNEHLIDMDHEASFFGAFLVG,
super-TRAIL AX-1630 protein sequence.
The linker sequence is underlined.
SEQ ID NO: 7
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGGGGGGVAAH
ITGTRGRSNTLSSPNSKNEKALGRKINSWESSRSGHSFLSNLHLR
NGELVIHEKGFYYIYSQTYFRFQEEIKENTKNDKQMVQYIYKYTS
YPDPILLMKSARNSCWSKDAEYGLYSIYQGGIFELKENDRIFVSV
TNEHLIDMDHEASFFGAFLVG,
super-TRAIL AX-1632 protein sequence.
The linker sequence is underlined.
SEQ ID NO: 8
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGGGGGGGVAA
HITGTRGRSNTLSSPNSKNEKALGRKINSWESSRSGHSFLSNLHL
RNGELVIHEKGFYYIYSQTYFRFQEEIKENTKNDKQMVQYIYKYT
SYPDPILLMKSARNSCWSKDAEYGLYSIYQGGIFELKENDRIFVS
VTNEHLIDMDHEASFFGAFLVG,
super-TRAIL AX-1618 protein sequence.
The linker sequence is underlined.
SEQ ID NO: 9
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGSGGGGSGGV
AAHITGTRGRSNTLSSPNSKNEKALGRKINSWESSRSGHSFLSNL
HLRNGELVIHEKGFYYIYSQTYFRFQEEIKENTKNDKQMVQYIYK
YTSYPDPILLMKSARNSCWSKDAEYGLYSIYQGGIFELKENDRIF
VSVTNEHLIDMDHEASFFGAFLVG,
super-TRAIL AX-1620 protein sequence.
The linker sequence is underlined.
SEQ ID NO: 10
VRERGPQRVAAHITGTRGRSNTLSSPNSKNEKALGRKINSWESSR
SGHSFLSNLHLRNGELVIHEKGFYYIYSQTYFRFQEEIKENTKND
KQMVQYIYKYTSYPDPILLMKSARNSCWSKDAEYGLYSIYQGGIF
ELKENDRIFVSVTNEHLIDMDHEASFFGAFLVGGGGSGGGGSGGG
SGGVAAHITGTRGRSNTLSSPNSKNEKALGRKINSWESSRSGHSF
LSNLHLRNGELVIHEKGFYYIYSQTYFRFQEEIKENTKNDKQMVQ
YIYKYTSYPDPILLMKSARNSCWSKDAEYGLYSIYQGGIFELKEN
DRIFVSVTNEHLIDMDHEASFFGAFLVG,
super-TRAIL AX-1606 protein sequence.
The linker sequence is underlined.
SEQ ID NO: 11
QRVAAHITGTRGRSNTLSSPNSKNEKALGRKINSWESSRSGHSFL
SNLHLRNGELVIHEKGFYYIYSQTYFRFQEEIKENTKNDKQMVQY
IYKYTSYPDPILLMKSARNSCWSKDAEYGLYSIYQGGIFELKEND
RIFVSVTNEHLIDMDHEASFFGAFLVGGGGSGGGGSGGQRVAAHI
TGTRGRSNTLSSPNSKNEKALGRKINSWESSRSGHSFLSNLHLRN
GELVIHEKGFYYIYSQTYFRFQEEIKENTKNDKQMVQYIYKYTSY
PDPILLMKSARNSCWSKDAEYGLYSIYQGGIFELKENDRIFVSVT
NEHLIDMDHEASFFGAFLVG,

### REFERENCES

1. Wiley, S. R., et al., Identification and characterization of a new member of the TNF family that induces apoptosis. Immunity, 1995. 3(6): p. 673-82.
2. Pitti, R. M., et al., Induction of apoptosis by Apo-2 ligand, a new member of the tumor necrosis factor cytokine family. J Biol Chem, 1996. 271(22): p. 12687-90.
3. Graves, J. D., et al., Apo2L/TRAIL and the death receptor 5 agonist antibody AMG 655 cooperate to promote receptor clustering and antitumor activity. Cancer Cell, 2014. 26(2): p. 177-89.
4. Kim, S. H., et al., Death induction by recombinant native TRAIL and its prevention by a caspase 9 inhibitor in primary human esophageal epithelial cells. J Biol Chem, 2004. 279(38): p. 40044-52.
5. Hutt, M., et al., Superior Properties of Fc-comprising scTRAIL Fusion Proteins. Mol Cancer Ther, 2017. 16(12): p. 2792-2802.

1. A fusion polypeptide comprising a first human TRAIL extracellular domain, a flexible linker, a second human TRAIL extracellular domain from N-terminus to C-terminus, wherein the fusion polypeptide forms a hexamer that contains two TRAIL trimers in solution.

2. The fusion polypeptide of claim 1, wherein the fusion polypeptide exhibits greatly improved biological activity to induce apoptosis in cancer cells compared with wild type human TRAIL.

3. The fusion polypeptide of claim 1, wherein the fusion polypeptide demonstrates an improved in vivo plasma half-life compared with the wild type human TRAIL.

4. The fusion polypeptide of claim 1, wherein the first human TRAIL extracellular domain and the second human TRAIL extracellular domain are selected from the group consisting of TRAIL residues 114-281, TRAIL residues 118-281, TRAIL residues 119-281, TRAIL residues 120-281, and TRAIL residues 122-281.

5. The fusion polypeptide of claim 1, wherein the first TRAIL extracellular domain and the second TRAIL extracellular domain are the same or different.

6. The fusion polypeptide of claim 1, wherein the fusion polypeptide comprises the amino acid sequence selected from SEQ ID NOs: 2 to 11, or comprises an amino acid sequence at least 90% identical thereto.

7. The fusion polypeptide of claim 1, wherein the fusion polypeptide is modified by PEGylation, lipidation or glycosylation.

8. A fusion protein comprising the fusion polypeptide of claim 1.

9. The fusion protein of claim 8, wherein the fusion protein comprises IgG Fc or HSA (human serum albumin).

10. A polynucleotide sequence encoding the fusion polypeptide of claim 1.

11. A pharmaceutical composition comprising the fusion polypeptide of claim 1 and a physiologically acceptable excipient.

12. A method for generating a more biologically potent TNF (tumor necrosis factor) superfamily member comprising connecting two TNF superfamily member molecules via a flexible linker to constitute a hexamer of two trimers of the TNF family member.

13. The method of claim 12, wherein the TNF family member is selected from the group consisting of TNF, 4-1BBL, Fas ligand, OX40L, CD40L, CD256, CD257, CD258 and GITRL.

14. (canceled)

15. (canceled)

16. A method of treating a cancer, comprising administrating to a subject in need thereof a therapeutically effective amount of the fusion polypeptide of claim 1.

17. The method of claim 16, wherein the cancer is a multiple myeloma or a lung cancer.

18. (canceled)

19. (canceled)

20. A pharmaceutical composition comprising the fusion protein of claim 8 and a physiologically acceptable excipient.

21. A method of treating a cancer, comprising administrating to a subject in need thereof a therapeutically effective amount of the fusion protein of claim 8.

22. A method of treating a cancer, comprising administrating to a subject in need thereof a therapeutically effective amount of the pharmaceutical composition of claim 11.
